(19) United States
(12) Patent Application Publication
(10) Pub. No.: US 2004/0037804 A1
(43) Pub. Date: Feb. 26, 2004

(54) Methods for treatment of asthma

(76) Inventor: Michael M. Grunstein, Merion, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/447,091

(22) Filed: May 28, 2003

Related U.S. Application Data
(60) Provisional application No. 60/384,246, filed on May 30, 2002.

(51) Int. Cl. A61K 38/20; A61K 31/573

(52) U.S. Cl. 424/85.1; 514/171; 424/85.2

(57) ABSTRACT

Methods combining glucocorticoids and cytokines for the treatment of inflammatory disorders are disclosed.

This application claims priority to U.S. Provisional Application No. 60/384,246 filed May 30, 2002, the entire disclosure of which is incorporated by reference herein.

### FIELD OF THE INVENTION

The present invention provides methods for administration of glucocorticoids in conjunction with inflammatory cytokines for treatment of a patient with an inflammatory disorder.

### BACKGROUND OF THE INVENTION

Several publications are referenced in this application in order to more fully describe the state of the art to which this invention pertains. The disclosure of each of these publications is incorporated by reference herein.

Asthma is characterized by hyper-responsiveness of the airways, episodic periods of bronchospasm, and chronic inflammation of the lungs. Obstruction of the airways is reversible with time or in response to drug therapies. Asthmatic patients may be hyper-reactive to a variety of naturally occurring stimuli, e.g., cold air, exercise, chemicals and allergens. The most common event initiating an asthmatic response is an immediate hypersensitivity to common allergens including ragweed pollen, grass pollen, various fungi, dust mites, cockroaches, and domestic animals. The symptoms of the disease include chest tightness, wheezing, shortness of breath, and coughing. Mild forms of the disease occur in up to 10% of the U.S. population, while the U.K., Australia and New Zealand report a higher prevalence of disease. Asthma incidence and mortality has been increasing worldwide, doubling over the past 20 years despite therapeutic intervention.

Allergy is characterized by increased blood serum levels of IgE antibody. Repeated exposure to allergens, which leads to sensitization, is normally required to elicit sufficient B cell production of IgE immunologically specific for a given allergen which triggers atopy and the subsequent asthmatic or allergic response. Exposure to allergens triggers B cells to produce antibodies which bind to the surface of mast cells. The cross-linking of two antibodies by the antigen causes a series of reactions leading to degranulation of mast cells and the release of a number of mediators which contribute to the inflammatory response, including, but not limited to: histamine, leukotrienes, prostaglandins, cytokines, and tryptase.

Eosinophils are also thought to contribute to the etiology of asthma. At the molecular level, eosinophil proliferation and differentiation are regulated by various cytokines, such as IL-3, IL-5, and GM-CSF. See Silberstein et al., Hematol. Oncol. Clin. North Am. 3: 511 (1989). These cytokines, as well as IFN-γ have been shown to prolong survival of eosinophils in vitro [Valerius et al. J. Immunol. 145: 2950 (1990)] and to augment eosinophil function [Rothenberg et al., J. Clin. Invest. 81: 1986 (1988); Fujisawa et al., Immunol. 144: 642 (1990); Silberstein et al., J. Immunol. 137: 3290 (1986)]. Other inflammatory cytokines, such as IL-1β and TNFα are also known to contribute to inflammatory responses.

The response of the airways to allergen is complex and often consists of an early asthmatic response (EAR) which peaks 20-30 minutes after exposure to the stimuli, is characterized by bronchoconstriction and normally resolves after 1.5 to 2 hours. The late asthmatic response (LAR) generally occurs 3-8 hours after initial exposure, and involves both bronchoconstriction and the development of inflammation and edema in the lung tissue. This inflammation often becomes chronic, with epithelial damage occurring and infiltration of the lungs with inflammatory cells such as eosinophils and neutrophils.

Corticosteroids (steroids) are known to inhibit the production of arachidonic acid metabolites (leukotrienes and prostaglandins) and cytokines by mast cells. Responses to inhaled steroids or systemic steroids can occur rapidly (within 4 hours) or may take several days depending on the severity of the disease state. Symptoms often return without regular chronic treatment. Side effects of inhaled steroids used on a continual basis include dysphonia, local irritation and oral candidiasis (a fungal infection). Higher doses of inhaled steroids cause suppression of the hypothalamic-pituitary-adrenocortical-(HPA)-axis which is responsible for the regulation of serum cortisol levels, metabolism, stress, CNS function and immunity. Continuous use of high dose inhaled steroids or oral steroids may induce more severe side effects, including: severe suppression of the HPA axis, which adversely affects the immune system; hypertension; osteoporosis; peptic ulcers; growth retardation in children; behavioral problems; reproductive problems; cataracts; and hematological disorders.

Moreover, many of the side effects of corticosteroid usage appear to be dose-dependent [Kimberly. Curr. Opin. Rheumatol. 4:325 (1992)]. Accordingly, methods and compositions that enable the use of a lower effective dosage of corticosteroids (referred to as the “steroid sparing effect”) would be highly desirable to avoid unwanted side effects.

As effective as inhaled steroids are, the above side effects limit their use. Combination therapy is often employed to minimize the amount of steroid administered, while reducing the severity of symptoms related to the disease and complications related to steroid treatment. Agents used in combination therapy are generally divided into the following categories: anti-inflammatory drugs (e.g., inhaled and oral steroids), bronchodilators, (e.g., β₂-agonists, xanthines, anticholinergics), and mediator inhibitors (e.g., cromolyns and leukotriene antagonists).

In general, however, moderate to severe asthma patients are often poorly served by the present armamentarium of drugs. Drugs that are safe may only be marginally effective and effective drugs can have unacceptable side effects. There is a significant need, therefore, for new methods directed to the efficacious treatment of patients afflicted with asthma and/or allergies. The present invention provides such methods and compositions for the treatment of patients in need thereof.

### SUMMARY OF THE INVENTION

In accordance with the present inventino, methods for the treatment of an inflammatory disease in a patient are provided. In one embodiment, the treatment comprises administering to the patient, a therapeutically effective amount of a glucocorticoid receptor agonist in conjunction with a therapeutically effective amount of an inflammatory cytokine, wherein the combinend administration reduces a plurality of symptoms of said inflammatory disease. The method of the invention may be used to advantage for the treatment of diseases associated with inflammation such as f asthma, rhinitis and atopic dermatitis.

Glucocorticoid receptor agonists useful in the inventive method include, without limitation, cortisone, hydrocortisone, prednisone, prednylidene, prednisolone, methylprednisolone, beclomethasone, flunisolide, triamcinolone, deflazacort, betamethasone and dexamethasone. Useful inflammatory cytokines include interleukin-1β, and tumor necrosis factor-α.

In yet another embodiment of the invention, a composition for administration to a patient in need thereof comprising a glucocorticoid receptor agonist and an inflammatory cytokine in a pharmaceutically acceptable carrier is provided.

### BRIEF DESCRIPTION OF THE DRAWINGS

FIG. 1 is a graph showing the effects of dexamethasone on Ach constrictor responsiveness is control and IL-1β/TNF-α-exposed airway smooth muscle (ASM) tissues.

FIG. 2 is a graph showing the effects of dexamethasone on relaxation responsiveness to isoproterenol in control and IL-1β/TNF-α-exposed ASM tissues.

FIG. 3 is a schematic drawing of the intracellular glucocorticoid signal transduction mechanism.

FIG. 4 is a graph showing the effect of IL-1β and TNF-α on dexamethasone induced changes in glucocorticoid receptor mRNA expression in human ASM cells.

FIGS. 5A and 5B are a pair of graphs showing real time quantitative PCR analysis of effects of dexamethasone on 11β-hydroxysteroid dehydrogenase (HSD) mRNA expression in human ASM cells.

FIG. 6 is a graph showing the effect of IL-1β/TNF-α on dexamethasone induced changes in mRNA expression of 11β-hydroxysteroid dehydrogenase (HSD) in cultured human ASM cells.

FIGS. 7A-7D are a series of immunofluorescent micrographs showing the effects of dexamethasone and IL-1β/TNF-α alone and in combination on cellular localization of glucocorticoid receptor α (Grα) in human ASM cells.

FIG. 8 is a schematic drawing of an assay method for detecting glucocorticoid/GRE signal transduction in ASM cells.

FIG. 9 is a graph showing the effects of IL-1β/TNF-α on dexamethasone induced secretion of alkaline phosphatase in pre-GRE-secAP transfected ASM cells.

### DETAILED DESCRIPTION OF THE INVENTION

The invention is directed to methods for using glucocorticoids in conjunction with inflammatory cytokines in the treatment of patients with inflammaotory disorders such as asthma, rhinitis and atopic dermatitis. The present inventor has discovered that using glucocorticoids in conjunction with inflammatory cytokines potentiates the anti-inflammatory properties of a glucocorticoid.

Isolated rabbit tracheal airway smooth muscle (ASM), an in vitro model system for studying asthma, was used to examine the effects of dexamethasone (DEX), interleukin-1β (IL-1β, and tumor necrosis factor-α (TNF-α) on constrictor and relaxation responses to acetylcholine (ACh) and isoproterenol (ISO), respectively. The physiological response of ASM to DEX, IL-1β, and TNF-α was determined using each of these modulators individually or in combination. IL-1β/TNF-α treated ASM exhibited increased constrictor responses to ACh and impaired relaxation responses to ISO, a pro-asthmatic like response pattern predicted following treatment with these inflammatory cytokines. As expected, pretreatment with DEX (1 μM) prevented the pro-asthmatic like response pattern following treatment with IL-1β/TNF-α. Additional studies demonstrated that treatment of cultured human airway smooth muscle cells (ASMs) with either DEX or IL-1β/TNF-α induced 1) increased mRNA expression of glucocorticoid (GC) receptor alpha (GRα); 2) enhanced mRNA expression of the GC converting enzyme 11β-hydroxysteroid dehydrogenase (11β-HSD-1; and 3) increased glucocorticoid (GC) response element (GRE) driven expression of the transfected reporter gene secretory alkaline phosphatase (AP) to which it was operably linked. These effects on the GC signaling system were potentiated in ASMs pretreated with the combination of DEX and IL-1β/TNF-α. Taken together, these results demonstrate that, while IL-1β and TNF-α exert pro-asthmatic effects on ASM responsiveness, these pro-inflammatory cytokines also serve to upregulate GC signal transduction in ASMs.

The effects of IL-1β and TNF-α in GC signaling pathways of ASMs were previously unappreciated. Moreover, the discovery that these inflammatory cytokines augment anti-inflammatory response pathways in ASMs is a surprising result in view of their other functional properties.

Based on these novel findings, the present inventor provides methods for the treatment of patients having inflammatory disorders with therapeutically effective amounts of glucocorticoids in conjunction with inflammatory cytokines. In a preferred embodiment, methods are provided for the treatment of patients having inflammatory disorders of the respiratory system, such as asthma and allergies. Glucocorticoids of utility in the methods of the present invention include, but are not limited to, glucocorticoids isolated from natural sources or synthetically produced. Synthetic glucocorticoids such as, for example, dexamethasone have been used to advantage in the treatment of asthmatic patients to reduce deleterious inflammatory responses of the respiratory tract for decades. Inflammatory cytokines of utility in the methods of the present invention include, but are not limited-to, IL-1β and TNF-α. The choice of glucocorticoid and inflammatory cytokine to be used and the dosage parameters for administration may be determined by a medical practitioner skilled in the art of pulmonary diseases. Such a skilled practitioner may also take into consideration other criteria which include, but are not limited to, the subject's condition, age, sex, the stage of disease, the subject's medical history.

Methods for the use of glucocorticoids in the treatment of human subjects with asthma and other inflammatory diseases have been previously disclosed in U.S. Pat. Nos. 6,046,185; 6,054,487; 6,071,910; 6,323,219; and 6,380,223, the entire contents of which are incorporated herein by reference. Methods for the use of IL-1β and TNF-α in the treatment of human subjects having a variety of different disorders have been previously disclosed in U.S. Pat. Nos. 6,274,552; 6,306,393; and 6,383,493, the entire contents of which are incorporated herein by reference.

The present invention also encompasses compositions comprising at least one glucocorticoid and at least one inflammatory cytokine in a pharmaceutically acceptable carrier, for example, HEPES buffered saline at a pH of about 7.8. Other pharmaceutically acceptable carriers which are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The compositions of the invention can be formulated neat or with a pharmaceutical carrier for administration to a patient in need thereof. The pharmaceutical carrier may be solid or liquid.

A solid carrier can include one or more substances which may also act as flavoring agent, lubricants, solubilizers, suspending agents, fillers, glidants, compression aids, binders or table-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers are used in preparing solutions, suspensions, emulsions, syrups, elixirs and pressurized composition. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agent, colors, viscosity regulators, stabilizers or osmo-regulators. Suitable examples of liquid carriers for oral and parenteral administration include water (partially containing additives as above, e.g., cellulose derivatives, preferably sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can also be an oily ester such as ethyl oleate and iopropyl myristate. Sterile liquid carriers are useful in sterile liquid form compositions for parenteral administration. The liquid carrier for pressurized compositions can be halogenated hydrocarbon or other pharmaceutically acceptable propellent.

Liquid pharmaceutical compositions which are sterile solutions or suspensions can be administered by, for example, intramuscular, intraperitoneal or subcutaneous injection. Alternatively they can be administered via an inhaler or a nebulizer. Sterile solutions can also be administered intravenously. The therapeutic agent can also be administered orally either in liquid or solid composition form.

The compositions of the invention may also be administered transdermally through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semipermeable membrane covering a reservoir containing the active ingredient with or without a carrier, or a matrix containing the active ingredient. Other occlusive devices are known in the literature.

Glucocorticosteroids (steroids) are by far the most effective drugs for the treatment of inflammatory diseases such as asthma and allergies. The glucocorticoid receptor (GR) is present in glucocorticoid responsive cells where it resides in the cytosol in an inactive state until it is stimulated by an agonist. Upon stimulation, the glucocorticoid receptor translocates to the cell nucleus wherein it binds to specific recognition motifs present in the DNA and/or protein(s). These binding events regulate the transcription of glucocorticoid responsive genes. The glucocorticoid receptor is also known to interact with the transcription factors, AP1 and NFκ-B. Such interactions result in inhibition of AP1- and NFκ-B-mediated transcription and are believed to be responsible for some of the anti-inflammatory activity of exogenously administered glucocorticoids. In addition, glucocorticoids may also exert physiologic effects independent of nuclear transcription.

Biologically relevant glucocorticoid receptor agonists include cortisol and corticosterone. Many synthetic glucocorticoid receptor agonists exist including, but not limited to cortisone, hydrocortisone, prednisone, prednylidene, prednisolone, methylprednisolone, beclomethasone, flunisolide, triamcinolone, deflazacort, betamethasone and dexamethasone. By definition, glucocorticoid receptor antagonists bind to the receptor and prevent glucocorticoid receptor agonists from binding and eliciting GR-mediated events, including transcription.

While the treatment of asthma is exemplified herein, the methods and compositions of the invention may be used to advantage in the treatment of other inflammatory disorders, such as rhinitis and atopic dermatitis.

The following example is provided to illustrate certain embodiments of the invention. It is not intended to limit the invention in any way.

### EXAMPLE I

Glucocorticoids (GCs) are commonly used to treat asthma due to their inherent anti-inflammatory properties. In accordance with the present invention, it has been discovered that pro-inflammatory cytokines upregulate the beneficial effects of glucocorticoid signaling in airway smooth muscle cells thereby providing a superior treatment modality for the treatment of asthma.

The following materials and methods are provided to facilitate the practice of the present invention.

Preparation and Treatment of Rabbit ASM Tissues

Following general anesthesia with xylazine (10 mg/kg) and ketamine 50 mg/kg), NZW rabbits were sacrificed by air embolism and the tracheae were removed, cleaned of loose connective tissue and epithelium, and divided into ring segments. Each alternate ring was incubated for 24 hours in either vehicle alone, the proinflammatory cytokines, IL-1β and TNF-α (20 and 10 ng/ml, respectively), dexamethasone (DEX; 1 μM), or the combination of DEX and IL-1β/TNF-α.

Human ASM Cell culture and Treatment

Cultured human ASM cells (Clonetics Corp., San Diego, Calif.) were grown in smooth muscle basal medium (SMBM) supplemented with 10% FBS, insulin (5 ng/ml), EGF (10 ng/ml; human recombinant) FGF (2 ng/ml, human recombinant), gentamicin (50 ng/ml), and amphotericin-B (50 ng/ml). After growing the cells to confluence, the cells were starved in unsupplemented SMBM for 24 hours, and then exposed for varying durations to either vehicle alone, IL-1β/TNF-α (5/10 ng/ml), DEX (1 μM), or the combination of DEX and IL-1β/TNF-α. Twenty-four hours thereafter, the cells were harvested and examined as described below.

Pharmacodynamic Studies of ASM Tissue Responsiveness

Following incubation, the ASM tissue segments were placed in organ baths containing modified Krebs Ringer solution, and aerated with 5% CO₂ in oxygen at 37° C. The tissues' isometric cholinergic contractility was subsequently assessed by cumulative administration of acetylcholine (ACh; 10⁻¹⁰ to 10⁻³ M). Thereafter, in separate studies, relaxation dose-response curves to isoproterenol (10⁻¹⁰-10⁻⁴ M) were generated in tissues initially half-maximally contracted with ACh.

RT-PCR

Total RNA was isolated from the ASM cells cultured under the experimental conditions described above. Typically, 2.5 μg of total RNA was reverse transcribed with an oligo dT primer using Superscript II (Invitrogen, Carlsbad, Calif.). The cDNA was then used as a template for PCR amplification with primers specific for the 11β-hydroxysteroid dehydrogenase (11β-HSD) isoforms, β-HSD-1 and 11β-HSD-2, and the glucocorticoid receptor (GR) isoforms, GRα and GRβ. PCR was performed with 35 cycles of denaturation at 94° C. for 30 sec, annealing at 60° C. for 30 sec, followed by elongation at 72° C. for 30 sec, and a final 72° C. extension for 7 min.

Real-Time Quantitative PCR

Real-time quantitative PCR was carried out using the ABI PRISM 7700 sequence detection system (Applied Biosystems, Foster City, Calif.) according to the manufacturer's protocol. The reaction volume used was 30 μl and cycling conditions included 40 cycles of 30 sec denaturation at 95° C. followed by 1 min annealing and elongation at 60° C. Fluorescence signals were detected for each cycle. To confirm the specificity of the amplifications, the PCR products were subjected to a melting curve analysis using the ABI PRISM 7000 sequence detection system (Applied Biosystems). The samples were first denatured at 95° C. and cooled to 63° C. The fluorescence signal was continuously monitored when the samples were slowly heated to 95° C. in about 20 min. The negative derivative of the fluorescence signal with respect to temperature was plotted against temperature to identify the melting temperature of the PCR products.

Assessment of GRE-Dependent Transcription Activation

ASM cells were grown to approximately 75% confluence and then co-transfected with a vector containing a secretory alkaline phosphatase (secAP) reporter driven by a glucocorticoid response element (GRE)-dependent promotor (pGRE-secAP) (Clontech), as well as pCMVSPORT-β-gal (Invitrogen, Carlsbad, Calif.) using TransIT-LT1 transfection reagent (Panvera, Madison, Wis.). After 24 hours, DEX with and without IL-1β/TNF-α was added to the culture media to incubate for an additional 24 hr. At the end of the incubation, culture media was collected to assay for secreted alkaline phosphatase (secAP) activity attributed to activation of transcription of the reporter by DEX and/or IL-1β/TNF-α. The secAP assay was carried out using the Great EscAPe SEAP chemiluminescence kit from Clontech. The measured levels of AP activity were then normalized to β-gal activity in order to correct for any variations caused by differences in transfection efficiency between the wells.

Indirect Immunofluorescence

ASM cells were grown on 4-chamber slides to about 85% confluence. DEX with and without IL-1β/TNF-α was then added to the cells. At various times thereafter, changes in intracellular localization of GR was examined by indirect immunofluoescence using a primary mouse anti-human GR antibody and a FITC-conjugated goat-anti-mouse IgG secondary antibody.

### Results

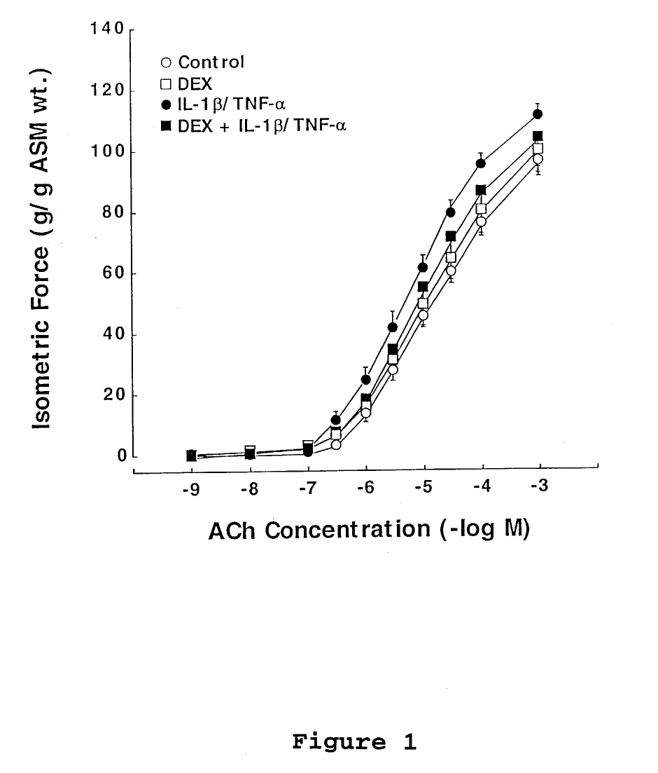

FIG. 1 is a graph showing the effects of dexamethasone on ACh constrictor responsiveness is control and IL-1β/TNF-α-exposed airway smooth muscle (ASM) tissues. Relative to control ASM, constrictor responses to ACh are increased in IL-1β/TNF-α exposed ASM tissues. Relative to control ASM, where DEX had no effect, heightened constrictor responses to ACh are inhibited in IL-1β/TNF-α exposed ASM tissues that were pretreated with DEX (1 μM).

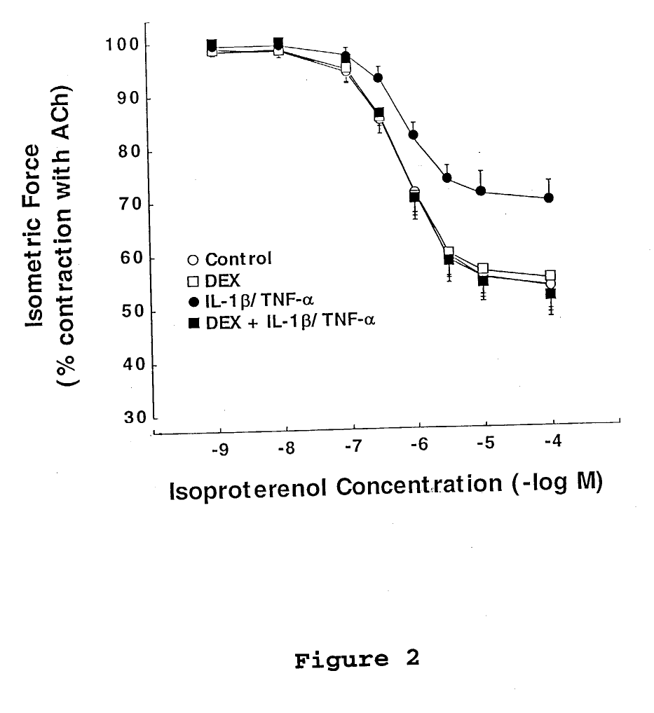

FIG. 2 is a graph showing the effects of dexamethasone on relaxation responsiveness to isoproterenol in control and IL-1β/TNF-α-exposed ASM tissues. Relative to control ASM, relaxation responses to isoproterenol are decreased in IL-1β/TNF-α exposed ASM tissues. Relative to control ASM, where DEX had no effect, impaired relaxation responses to isoproterenol are prevented in IL-1β/TNF-α exposed ASM tissues that were pretreated with DEX (1 μM).

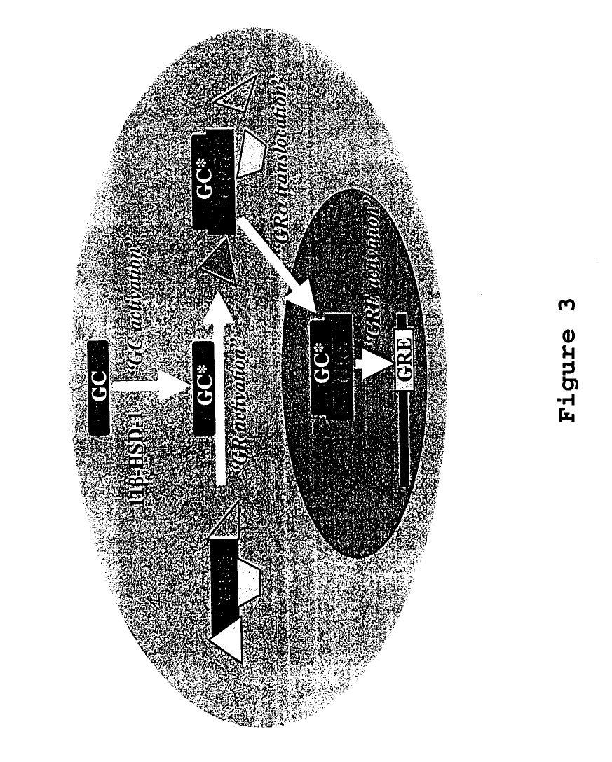

FIG. 3 is a schematic drawing of the intracellular glucocorticoid signal transduction mechanism. The diagram shows the conversion of inactive GC into bioactive GC* by the cytosolic enzyme 11β-hydroxysteroid dehydrogenase-type 1 (11β-HSD-1). Following GC receptor activation, translocation of the GC*/GRα complex to the nucleus occurs. This in turn is followed by activation of genes containing glucocorticoid response elements which are bound by the translocated GC*/GRα complex.

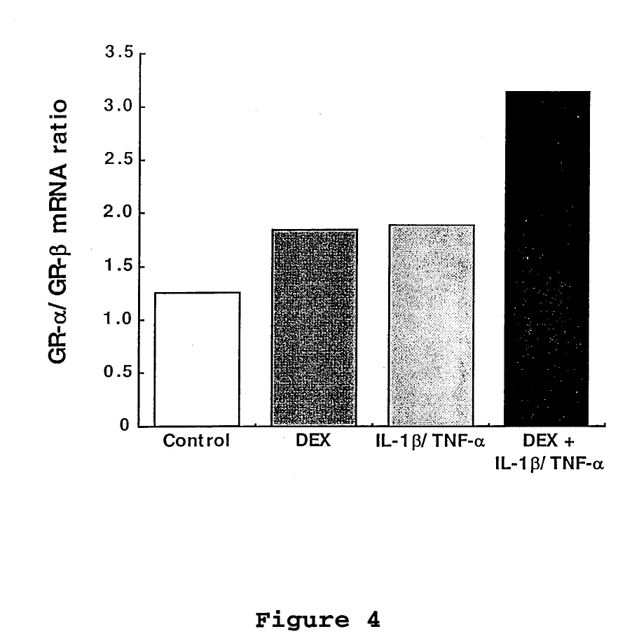

FIG. 4 is a graph showing the effect of IL-1β and TNF-α on dexamethasone induced changes in glucocorticoid receptor mRNA expression in human ASM cells. Relative to control (untreated) ASM cells, the ratio of GRα/GRβ isoforms (i.e., “bio-activating”/“bio-inactivating”) is moderately increased (i.e., by 50%) in ASM cells treated with either DEX or IL-1β/TNF-α alone. In combination, DEX+IL-1β/TNF-α produced a potentiated increase in the Grα/GRβ ratio (i.e., by 150%) in ASM cells.

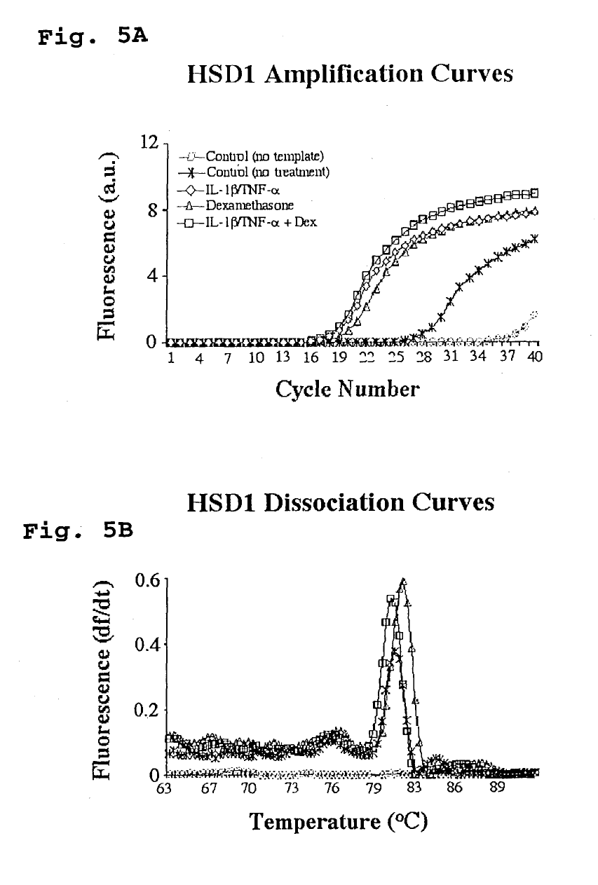

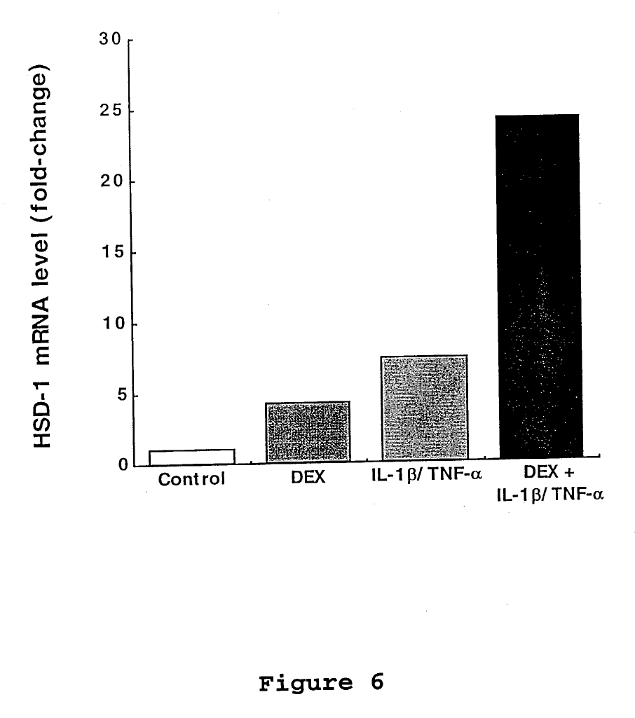

FIGS. 5A and 5D are a pair of graphs showing real time quantitative PCR analysis of effects of dexamethasone on 11β-hydroxysteroid dehydrogenase (HSD) mRNA expression in human ASM cells. FIG. 6 is a graph showing the effect of IL-1β/TNF-α on dexamethasone induced changes in mRNA expression of 11β-hydroxysteroid dehydrogenase (HSD) in cultured human ASM cells. As quatified by real time PCR, relative to control (untreated) ASM cells, HSD-1 mRNA expression is moderately increased in ASM cells treated with either DEX or IL-1β/TNF-α alone. In combination, DEX or IL-1β/TNF-α produced a markedly potentiated increase in HSD-1 mRNA expression in ASM cells. In contrast, neither DEX or IL-1β/TNF-α alone or in combination had a systematic effect on HSD-2 mRNA expression (data not shown).

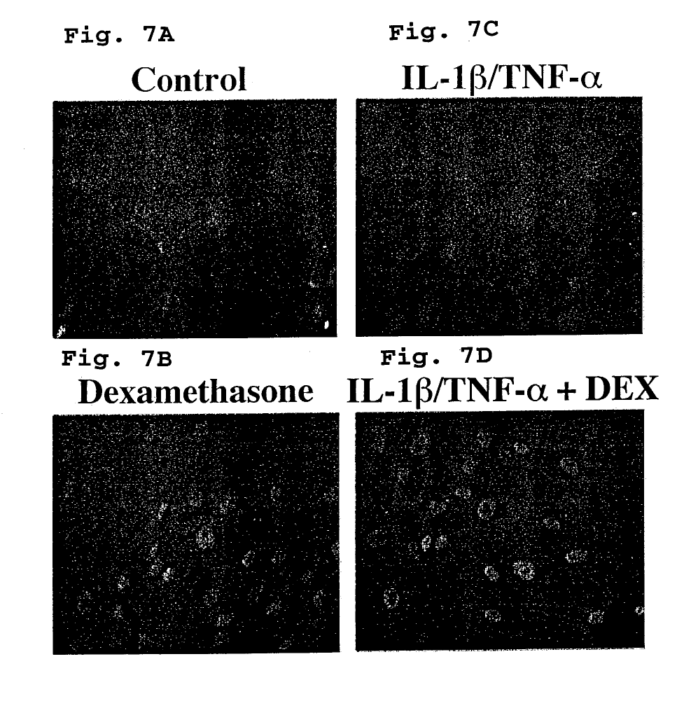

FIGS. 7A-7D are a series of immunofluorescent micrographs showing the effects of dexamethasone and IL-1β/TNF-α alone and in combination on cellular localization of glucocorticoid receptor α (Grα) in human ASM cells. The data show that cytosolic and intra-nuclear immunofluorescent staining of the glucocorticoid receptor (GR) is present in ASM cells under control conditions. Intra-nuclear detection of GR is relatively increased in ASM cells treated with either DEX or IL-1β/TNF-α. Notably, intra-nuclear detection of GR is most enhanced in ASM cells treated with a combination of DEX and IL-1/TNF-α.

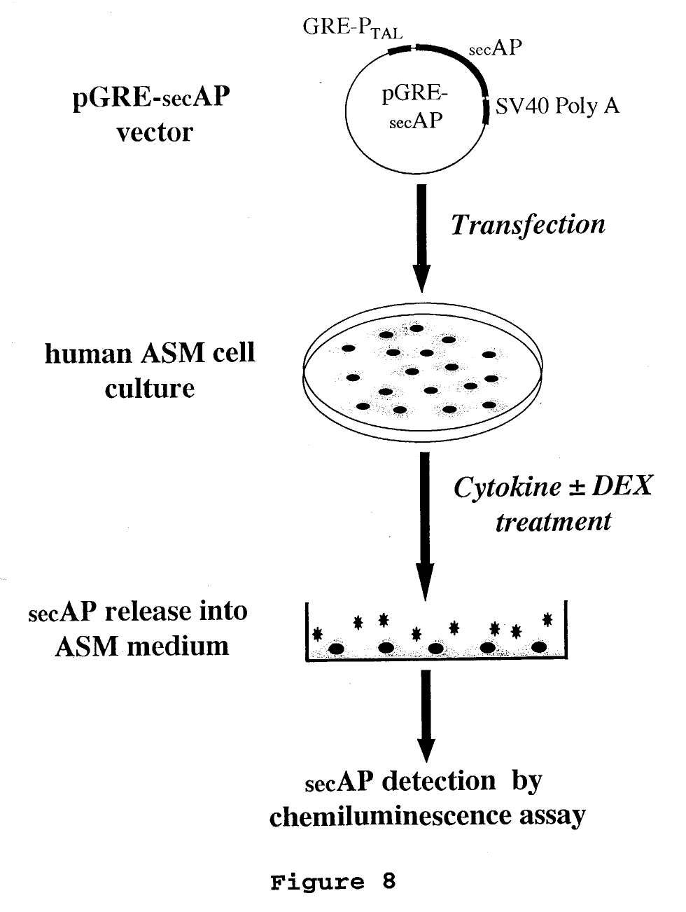

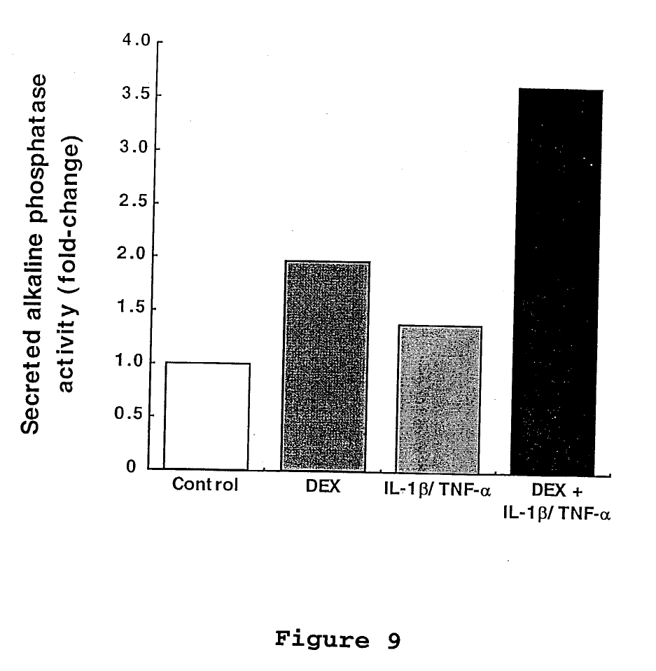

FIG. 8 is a schematic drawing of an assay method for detecting glucocorticoid/GRE signal transduction in ASM cells. FIG. 9 is a graph showing the effects of IL-1β/TNF-α on dexamethasone induced secretion of alkaline phosphatase in pre-GRE-secAP transfected ASM cells. As quantified by chemiluminescence assay, relative to control, untreated ASM cells, secretion of alkaline phosphatase is enhanced in ASM cells treated with either DEX or IL-1β/TNF-α alone. In combination DEX+IL-1β/TNF-α produced a potentiated increase in alkaline phosphatase secretion from ASM cells.

### Discussion

While the glucocorticoid, dexamethasone (DEX) has no effect in naive ASM, DEX prevents the pro-asthmatic-like changes in constrictor and relaxant responsiveness induced in ASM exposed to IL-1β/TNF-α.

Additionally, treatment with DEX increases mRNA expression of 11β-HSD-1, the mRNA ratio of GRα/GRβ, and nuclear translocation of Grα in cultured human ASM cells. Surprisingly, the above effects of DEX in ASM cells are potentiated in the presence of IL-1β/TNF-α. The above cooperative effects of DEX and IL-1β/TNF-α are reflected by potentiated net glucocorticoid/GRE-mediated signal transduction in human ASM cells

In conclusion, the present findings support the novel concept that the efficacy of glucocorticoids in the treatment of asthma is attributed, at least in part, to the cooperative (“permissive”) effects of certain proinflammatory/pro-asthmatic cytokines on activation of the glucocorticoid signal transduction mechanism in airway smooth muscle. Accordingly, combination treatments using glucocorticoids in conjunction with certain inflammatory cytokines provides a therapeutically superior approach for alleviating the symptoms of inflammatory disorders such as asthma, rhinitis and atopic dermatitis.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for the treatment of an inflammatory disease in a mammal, said treatment comprising:
administering to said mammal a therapeutically effective amount of a glucocorticoid receptor agonist in conjunction with a therapeutically effective amount of an inflammatory cytokine, wherein said administration reduces a plurality of symptoms of said inflammatory disease.

2. The method as claimed in claim 1, wherein said inflammatory disease is selected from the group consisting of asthma, rhinitis and atopic dermatitis.

3. The method as claimed in claim 1, wherein said glucocorticoid receptor agonist is a compound selected from the group consisting of cortisone, hydrocortisone, prednisone, prednylidene, prednisolone, methylprednisolone, beclomethasone, flunisolide, triamcinolone, deflazacort, betamethasone and dexamethasone.

4. The method as claimed in claim 1, wherein said inflammatory cytokine is selected from the group consisting of interleukin-1β, and tumor necrosis factor-α.

5. A method as claimed in claim 1, wherein said inflammatory cytokine is selected from the group consisting of cortisone, hydrocortisone, prednisone, prednylidene, prednisolone, methylprednisolone, beclomethasone, flunisolide, triamcinolone, deflazacort, betamethasone and dexamethasone and wherein said inflammatory cytokine is selected from the group consisting of interleukin-1β, and tumor necrosis factor-α.

6. A composition for administration to a patient in need thereof comprising a glucocorticoid receptor agonist and an inflammatory cytokine in a pharmaceutically acceptable carrier.

7. The composition of claim 6, wherein said glucocorticoid receptor agonist is a compound selected from the group consisting of cortisone, hydrocortisone, prednisone, prednylidene, prednisolone, methylprednisolone, beclomethasone, flunisolide, triamcinolone, deflazacort, betamethasone and dexamethasone.

8. The composition of claim 6, wherein said inflammatory cytokine is selected from the group consisting of interleukin-1β, and tumor necrosis factor-α.

9. The composition of claim 6, wherein said glucocorticoid receptor agonist is a compound selected from the group consisting of cortisone, hydrocortisone, prednisone, prednylidene, prednisolone, methylprednisolone, beclomethasone, flunisolide, triamcinolone, deflazacort, betamethasone and dexamethasone and wherein said inflammatory cytokine is selected from the group consisting of interleukin-1β and tumor necrosis factor-α.
